(19) United States
(12) Patent Application Publication
(10) Pub. No.: US 2024/0252832 A1
(43) Pub. Date: Aug. 1, 2024

(54) IMPLANTABLE MEDICAL DEVICE, IMPLANT COMMUNICATION SYSTEM AND METHOD FOR UPDATING AN EXECUTABLE CODE OF AN IMPLANTABLE MEDICAL DEVICE

(71) Applicant: BIOTRONIK SE & Co. KG, Berlin (DE)

(72) Inventor: Thomas DOERR, Berlin (DE)

(73) Assignee: BIOTRONIK SE & Co. KG, Berlin (DE)

(21) Appl. No.: 18/560,494

(22) PCT Filed: Jun. 2, 2022
(86) PCT No.: PCT/EP2022/065040

§ 371 Date: Nov. 13, 2023

(30) Foreign Application Priority Data
Jun. 29, 2021 (EP) 21182298.6

(51) Int. Cl. A61N 1/372

(52) U.S. Cl. CPC A61N 1/37254; A61N 1/37217; A61N 1/37235; A61N 1/37264; A61N 1/37276

(57) ABSTRACT

The invention relates to an implantable medical device, in particular a diagnostic monitoring device, a pacemaker, a defibrillator and/or a neuro-stimulator, comprising a diagnostic and/or therapeutic control unit, a wireless communication interface, operating in a frequency band and/or using a communication protocol supported by consumer mobile communications devices, in particular smartphones and/or tablet computing devices, a first memory area, in which executable code for controlling the diagnostic and/or therapy control unit is stored, and a second memory area, in which executable code for controlling the wireless communication interface is stored, wherein the software and/or hardware of the wireless communication interface is configured to update the executable code of the wireless communication interface via the wireless communication interface. The invention further relates to an implant communication system, a method for updating an executable code of an implantable medical device and a computer program.

### CROSS-REFERENCE TO RELATED APPLICATIONS

This application is the United States National Phase under 35 U.S.C. § 371 of PCT International Patent Application No. PCT/EP2022/065040, filed on Jun. 2, 2022, which claims the benefit of European Patent Application No. 21182298.6, filed on Jun. 29, 2021, the disclosures of which are hereby incorporated by reference herein in their entireties.

### TECHNICAL FIELD

The present invention relates to an implantable medical device, in particular a diagnostic monitoring device, a pacemaker, a defibrillator and/or a neuro-stimulator. The present invention further relates to an implant communication system.

Moreover, the present invention relates to a computer implemented method for updating an executable code of an implantable medical device, in particular a diagnostic monitoring device, a pacemaker, a defibrillator and/or a neuro-stimulator.

### BACKGROUND

Active electronic implants are customarily equipped with Bluetooth Low Energy radios to enable communication with commercially available smartphones and tablets. Currently, firmware updates of implantable medical devices including the Bluetooth stack update are performed on implants with a programming device.

The disadvantage of the current solution of updating the Bluetooth stack by means of a programming device is that the implant carrier has to be called in for the update thus resulting in significant costs and effort for the update. In addition the time period for an area-wide update of all implants is very long. In the case of urgent updates, an official recall might have to take place in order to update all implants quickly.

The present disclosure is directed toward overcoming one or more of the above-mentioned problems, though not necessarily limited to embodiments that do.

### SUMMARY

It is therefore an object of the present invention to provide an improved implantable medical device the Bluetooth stack of which can be updated without having to call in the patient.

At least the object is solved by an implantable medical device, in particular a diagnostic monitoring device, a pacemaker, a defibrillator and/or a neuro-stimulator having the features of claim 1.

At least the object is furthermore solved by an implant communication system having the features of claim 8.

In addition, at least the object is solved by a computer implemented method for updating an executable code of an implantable medical device, in particular a diagnostic monitoring device, a pacemaker, a defibrillator and/or a neuro-stimulator having the features of claim 11.

Moreover, at least the object is solved by a computer program of claim 12.

The present invention provides an implantable medical device, in particular a diagnostic monitoring device, a pacemaker, a defibrillator and/or a neuro-stimulator.

The implantable medical device comprises a diagnostic and/or therapeutic control unit and a wireless communication interface, operating in a frequency band and/or using a communication protocol supported by consumer mobile communications devices, in particular smartphones and/or tablet computing devices.

Furthermore, the implantable medical device comprises a first memory area, in which executable code for controlling the diagnostic and/or therapy control unit is stored. In addition, the implantable medical device comprises a second memory area, in which executable code for controlling the wireless communication interface is stored, wherein the software and/or hardware of the wireless communication interface is configured to update the executable code of the wireless communication interface via the wireless communication interface.

The present invention further provides an implant communication system, comprising the implantable medical device according to the present invention, a programmer configured to communicate with the implantable medical device using inductive communication or medical RF communication and a consumer mobile communication device, in particular a smartphone or tablet computing device, configured to communicate with the implantable medical device in a frequency band and/or using a communication protocol supported by the consumer mobile communication device.

Moreover, the present invention further provides a computer implemented method for updating an executable code of an implantable medical device, in particular a diagnostic monitoring device, a pacemaker, a defibrillator and/or a neuro-stimulator.

The method comprises providing a diagnostic and/or therapeutic control unit and providing a wireless communication interface operating in a frequency band and/or using a communication protocol supported by consumer mobile communications devices, in particular smartphones and/or tablet computing devices.

Furthermore, the method comprises storing executable code for controlling the diagnostic and/or therapy control unit in a first memory area and storing executable code for controlling the wireless communication interface in a second memory area.

The method moreover comprises updating the executable code of the wireless communication interface by means of a software and/or hardware configuration of the wireless communication interface via the wireless communication interface.

The Bluetooth stack, i.e., the software that controls the Bluetooth communication in the implant is updated regularly. According to the present invention the update can be performed wirelessly such that the patient does not have to be called in.

The update of the communication unit of the implant thus does not have any influence on the therapy control of the implant. The first memory area can hence be updated without the second memory area being affected by said update. This is a risk-mitigating measure to prevent an update of the Bluetooth stack from negatively influencing the therapy function of the implant.

An example of a diagnostic device is a heart rhythm monitor, which in the event of abnormalities makes ECG recordings that can later be evaluated by a physician.

An example of a therapy device is a cardiac pacemaker which has no diagnostic function but provides solely therapy. Many devices however have a diagnostic as well as a therapeutic function.

According to an aspect of the present invention, the implantable medical device comprises at least one further wireless communication interface configured to update the executable code for controlling the diagnostic and/or therapeutic control unit, wherein the at least one further wireless communication interface comprises an inductive communication interface and/or a MICS band communication interface.

Thus, the diagnostic and/or therapeutic control unit can be safely updated via the at least one further wireless communication interface which is not accessible via the consumer mobile communication device.

According to a further aspect of the present invention, the at least one further wireless communication interface is configured to authenticate an update of the executable code for controlling the diagnostic and/or therapeutic control unit, wherein an authentication of the update is performed via the inductive communication interface. The authentication of the update is thus either performed by a physician or by the patient, wherein said update cannot be performed via Bluetooth connectivity such that an unintended and/or an authenticated update attempt is effectively inhibited.

According to a further aspect of the present invention, the implantable medical device is configured to use a first authentication mechanism for authenticating an update of the executable code for controlling the diagnostic and/or therapeutic control unit, and wherein the implantable medical device is configured to use a second authentication mechanism for authenticating an update of the executable code for controlling the wireless communication interface.

By using the first authentication mechanism for authenticating an update of the executable code for controlling the diagnostic and/or therapeutic control unit and the second authentication mechanism for authenticating an update of the executable code for controlling the wireless communication interface updates of the diagnostic and/or therapeutic control unit and the wireless communication interface are effectively separately, i.e., independently authenticated, thus adding an additional layer of security.

According to a further aspect of the present invention, the diagnostic and/or therapeutic control unit is configured to prohibit an update of the executable code for controlling the wireless communication interface if a predetermined condition is met, in particular if higher-priority system tasks are pending and/or if an unauthenticated update attempt is performed. The diagnostic and/or therapeutic control unit is thus configured to override update requests for updating in the executable code for controlling the wireless communication interface if said predetermined condition is met.

According to a further aspect of the present invention, the wireless communication interface uses an authentication and/or data encryption method supported by consumer mobile communications devices, in particular smartphones and/or tablet computing devices. This advantageously enables the usage of existing standards for authentication and/or data encryption method supported by consumer mobile communications devices ensuring a secure connection and data transferred to the implantable medical device.

According to a further aspect of the present invention, the wireless communication interface is a, in particular software-controlled, Bluetooth, Bluetooth Low Energy and/or Bluetooth Mesh communication interface. This ensures compatibility with a wide range of consumer mobile communications devices.

According to a further aspect of the present invention, the consumer mobile communication device is configured to authenticate an update of the executable code for controlling the wireless communication interface using an app connected to the implantable medical device. The updates of the executable code for controlling the wireless communication interface can thus be easily performed by the patient using the app installed in the consumer mobile communications device.

According to a further aspect of the present invention, the programmer is configured to authenticate an update of the executable code for controlling the diagnostic and/or therapeutic control unit. The authentication of the update is thus either performed by a physician or by the patient, wherein said update cannot be performed via Bluetooth connectivity such that an unintended and/or an authenticated update attempt is effectively inhibited.

The herein described features of the implantable medical device configured to exchange data with an external communication device are also disclosed for the computer implemented method for updating an executable code of an implantable medical device and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The present invention is explained in more detail below using exemplary embodiments, which are specified in the schematic figures of the drawings, in which:

FIG. 1 shows a schematic view of an implantable medical device according to a preferred embodiment of the present invention;

FIG. 2 shows a schematic view of an implant communication system according to the preferred embodiment of the present invention; and

FIG. 3 shows a flowchart of a computer implemented method for transferring data between an implantable medical device and an external communication device according to the preferred embodiment of the present invention.

### DETAILED DESCRIPTION

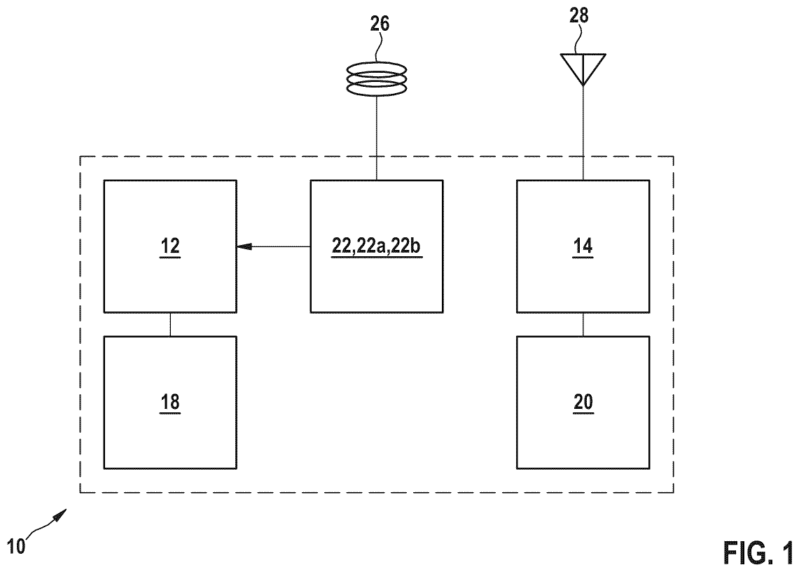

The implantable medical device 10 of FIG. 1, in particular a diagnostic monitoring device, a pacemaker, a defibrillator and/or a neuro-stimulator comprises a diagnostic and/or therapeutic control unit 12 and a wireless communication interface 14, operating in a frequency band and/or using a communication protocol supported by consumer mobile communications devices 16, in particular smartphones and/or tablet computing devices.

The implantable medical device 10 further comprises a first memory area 18, in which executable code for controlling the diagnostic and/or therapy control unit 12 is stored. Specifically, the implantable medical device 10 comprises a software-controlled therapy controller, whose executable code is stored in the first memory area 18.

The implantable medical device 10 moreover comprises a second memory area 20, in which executable code for controlling the wireless communication interface 14 is stored. Specifically, the implantable medical device 10 comprises a software-controlled Bluetooth low energy BLE radio, the executable code of which is stored in a second memory area 20. Alternatively, or in addition, the wireless communication interface 14 may also support Bluetooth and/or Bluetooth Mesh radio.

The BLE radio is connected to a second antenna 28. Both memory areas are separated from each other in the architecture of the memory access in such a way that at least the second memory area 20, i.e., the BLE radio cannot directly write-access the first memory area 18.

The software and/or hardware of the wireless communication interface 14 is configured to update the executable code of the wireless communication interface 14 via the wireless communication interface 14.

The implantable medical device 10 comprises at least one further wireless communication interface 22 configured to update the executable code for controlling the diagnostic and/or therapeutic control unit 12, wherein the at least one further wireless communication interface 22 comprises an inductive communication interface 22a and/or a MICS band communication interface 22b. The diagnostic and/or therapeutic control unit 12 is connected to a first antenna 26 and is configured to exchange authentication information between an authorized external device and the therapy controller.

The at least one further wireless communication interface 22 is configured to authenticate an update of the executable code for controlling the diagnostic and/or therapeutic control unit 12, wherein an authentication of the update is performed via the inductive communication interface 22a.

The patient thus authorizes the update via a separate device, i.e., via the inductive communication interface 22a.

In practice, a message is displayed on the patient's consumer mobile communication device 16, e.g., “there is an update available for your implant. Please authorize the update with your patient device.” The patient device, i.e., the inductive communication interface 22a is placed in the vicinity of the implant. Authentication then takes place in the implant so that the implant accepts the update.

The implantable medical device 10 is configured to use a first authentication mechanism for authenticating an update of the executable code for controlling the diagnostic and/or therapeutic control unit 12. In addition, the implantable medical device 10 is configured to use a second authentication mechanism for authenticating an update of the executable code for controlling the wireless communication interface 14.

The diagnostic and/or therapeutic control unit 12 is configured to prohibit an update of the executable code for controlling the wireless communication interface 14 if a predetermined condition is met, in particular if higher-priority system tasks are pending and/or if an unauthenticated update attempt is performed.

The wireless communication interface 14 uses an authentication and/or data encryption method supported by consumer mobile communications devices 16, in particular smartphones and/or tablet computing devices.

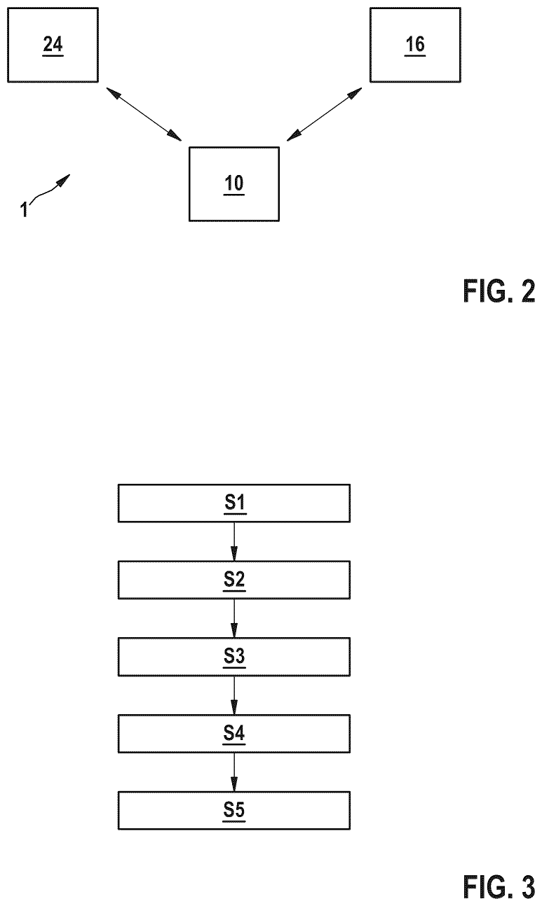

FIG. 2 shows a schematic view of an implant communication system 1 according to the preferred embodiment of the present invention.

The implant communication system 1 comprises the implantable medical device 10 according to the present invention.

Furthermore, the implant communication system 1 comprises a programmer 24 configured to communicate with the implantable medical device 10 using inductive communication or medical RF communication.

In addition, the implant communication system 1 comprises a consumer mobile communication device 16, in particular a smartphone or tablet computing device, configured to communicate with the implantable medical device 10 in a frequency band and/or using a communication protocol supported by the consumer mobile communication device 16.

The consumer mobile communication device 16 is configured to authenticate an update of the executable code for controlling the wireless communication interface 14 using an app connected to the implantable medical device 10. Furthermore, the programmer 24 is configured to authenticate an update of the executable code for controlling the diagnostic and/or therapeutic control unit 12.

FIG. 3 shows a flowchart of a computer implemented method for transferring data between an implantable medical device 10 and an external communication device according to the preferred embodiment of the present invention.

The computer implemented method for updating an executable code of an implantable medical device 10, in particular a diagnostic monitoring device, a pacemaker, a defibrillator and/or a neuro-stimulator, comprises providing S1 a diagnostic and/or therapeutic control unit 12 and providing S2 a wireless communication interface 14, operating in a frequency band and/or using a communication protocol supported by consumer mobile communications devices 16, in particular smartphones and/or tablet computing devices.

Furthermore, the method comprises storing S3 executable code for controlling the diagnostic and/or therapy control unit 12 in a first memory area 18 and storing S4 executable code for controlling the wireless communication interface 14 in a second memory area 20.

Moreover, the method comprises updating S5 the executable code of the wireless communication interface 14 by means of a software and/or hardware configuration of the wireless communication interface 14 via the wireless communication interface 14.

### Further Embodiments

A computer implemented method for updating a medical device implanted into the human or animal body, in particular a diagnostic monitoring device, a pacemaker, a defibrillator and/or a neuro-stimulator, comprising providing a diagnostic and/or therapeutic control unit 12 and providing a wireless communication interface 14, operating in a frequency band and/or using a communication protocol supported by consumer mobile communications devices 16, in particular smartphones and/or tablet computing devices.

The method further comprises storing executable code for controlling the diagnostic and/or therapy control unit 12 in a first memory area 18 and storing executable code for controlling the wireless communication interface 14 in a second memory area 20.

Moreover, the method comprises updating the executable code of the wireless communication interface 14 by means of a software and/or hardware configuration of the wireless communication interface 14 via the wireless communication interface 14.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE SIGNS

1 system
10 implantable medical device
12 diagnostic and/or therapeutic control unit
14 wireless communication interface
16 consumer mobile communication device
18 first memory area
20 second memory area
22 further wireless communication interface
22a inductive communication interface
22b MICS band communication interface
24 programmer
26 first antenna
28 second antenna
S1-S5 method steps

1. Implantable medical device, in particular a diagnostic monitoring device, a pacemaker, a defibrillator and/or a neuro-stimulator, comprising:
a diagnostic and/or therapeutic control unit;
a wireless communication interface, operating in a frequency band and/or using a communication protocol supported by consumer mobile communications devices, in particular smartphones and/or tablet computing devices;
a first memory area, in which executable code for controlling the diagnostic and/or therapy control unit is stored; and
a second memory area, in which executable code for controlling the wireless communication interface is stored, wherein the software and/or hardware of the wireless communication interface is configured to update the executable code of the wireless communication interface via the wireless communication interface.

2. Implantable medical device of claim 1, comprising at least one further wireless communication interface configured to update the executable code for controlling the diagnostic and/or therapeutic control unit, wherein the at least one further wireless communication interface comprises an inductive communication interface and/or a MICS band communication interface.

3. Implantable medical device of claim 2, wherein the at least one further wireless communication interface is configured to authenticate an update of the executable code for controlling the diagnostic and/or therapeutic control unit, wherein an authentication of the update is performed via the inductive communication interface.

4. Implantable medical device of claim 1, wherein the implantable medical device is configured to use a first authentication mechanism for authenticating an update of the executable code for controlling the diagnostic and/or therapeutic control unit, and wherein the implantable medical device is configured to use a second authentication mechanism for authenticating an update of the executable code for controlling the wireless communication interface.

5. Implantable medical device of claim 1, wherein the diagnostic and/or therapeutic control unit is configured to prohibit an update of the executable code for controlling the wireless communication interface if a predetermined condition is met, in particular if higher-priority system tasks are pending and/or if an unauthenticated update attempt is performed.

6. Implantable medical device of claim 1, wherein the wireless communication interface uses an authentication and/or data encryption method supported by consumer mobile communications devices, in particular smartphones and/or tablet computing devices.

7. Implantable medical device of claim 1, wherein the wireless communication interface is a, in particular software-controlled, Bluetooth, Bluetooth Low Energy and/or Bluetooth Mesh communication interface.

8. Implant communication system, comprising:
the implantable medical device of claim 1;
a programmer configured to communicate with the implantable medical device using inductive communication or medical RF communication; and
a consumer mobile communication device, in particular a smartphone or tablet computing device, configured to communicate with the implantable medical device in a frequency band and/or using a communication protocol supported by the consumer mobile communication device.

9. Implant communication system of claim 8, wherein the consumer mobile communication device is configured to authenticate an update of the executable code for controlling the wireless communication interface using an app connected to the implantable medical device.

10. Implant communication system of claim 8, wherein the programmer is configured to authenticate an update of the executable code for controlling the diagnostic and/or therapeutic control unit.

11. Computer implemented method for updating an executable code of an implantable medical device, in particular a diagnostic monitoring device, a pacemaker, a defibrillator and/or a neuro-stimulator, comprising the steps of:
providing a diagnostic and/or therapeutic control unit;
providing a wireless communication interface, operating in a frequency band and/or using a communication protocol supported by consumer mobile communications devices, in particular smartphones and/or tablet computing devices;
storing executable code for controlling the diagnostic and/or therapy control unit in a first memory area;
storing executable code for controlling the wireless communication interface in a second memory area; and
updating the executable code of the wireless communication interface by means of a software and/or hardware configuration of the wireless communication interface via the wireless communication interface.

12. Computer program with program code to perform the method of claim 11 when the computer program is executed on a computer.
